(19) United States
(12) Patent Application Publication
(10) Pub. No.: US 2017/0319519 A1
(43) Pub. Date: Nov. 9, 2017

(54) COMBINATION FOR THE TREATMENT OF CONDITIONS INVOLVING MUSCULAR PAIN

(71) Applicant: Spherium Biomed S.L., Barcelona (ES)

(72) Inventors: Pablo Jose Llorens Bullemore, Barcelona (ES); Ramon Bosser Artal, Barcelona (ES); Wilmar Castillo Ávila, Barcelona (ES); Benjamin Santos Lobo, Barcelona (ES)

(21) Appl. No.: 15/523,299

(22) PCT Filed: Nov. 2, 2015
(86) PCT No.: PCT/EP2015/075417

§ 371 Date: Apr. 28, 2017

(30) Foreign Application Priority Data
Oct. 31, 2014 (EP) 14382429.0
Dec. 23, 2014 (ES) P 201431929

(51) Int. Cl. A61K 31/192; A61K 45/06; A61K 33/06; A61K 31/375; A61K 9/00; A61K 9/14; A61K 47/26; A61K 31/5415; A61K 31/00

(52) U.S. Cl. CPC A61K 31/192; A61K 47/26; A61K 45/06; A61K 33/06; A61K 31/5415; A61K 31/375; A61K 9/0095; A61K 9/146; A61K 31/00; A61K 9/00

(57) ABSTRACT

The present invention is related to a combination comprising a COX inhibitor, a Mg²⁺ source, and ascorbic acid or a stereoisomer or a pharmaceutically acceptable salt thereof, uses and pharmaceutical composition thereof.

### FIELD OF THE INVENTION

The present invention relates to a combination of pharmaceutical ingredients and its use in the treatment or prevention of conditions involving muscular pain, optionally accompanied with muscular imbalances, such as pain accompanying muscle contracture, pain accompanying muscle overload following exercise, muscle pain during episodes of fibromyalgia and, in particular, temporomandibular joint disorder.

### BACKGROUND OF THE INVENTION

The use of cyclooxygenase inhibitors also known as COX inhibitors as analgesic or pain killers is well known in the art and its use includes the mitigation of different types of pain, including muscular pain. However, in the case of muscular pain this symptom is very often accompanied with one or more muscular imbalances such as muscle contractures, muscle overload, muscle micro-tears, and muscle cramps. The use of COX inhibitors alone reduces muscular pain, but often it is not useful to reduce or revert the symptoms associated with muscular imbalances in conditions involving muscular pain accompanied with said muscular imbalances, such as pain accompanying muscle contracture, pain accompanying muscle overload following exercise, muscle pain during episodes of fibromyalgia and, in particular, temporomandibular joint disorder.

The treatment of muscular pain with COX inhibitors such as ibuprofen is known. For example, US 2002/0022058 A1 discloses, in its Example 18, a composition comprising 200 mg ibuprofen, 100 mg magnesium, and 60 mg vitamin C.

In the light of this prior art, the present invention addresses the problem of providing an efficacious treatment for muscular pain, in particular effective pain reduction and restoration of temporomandibular joint function.

### SUMMARY OF THE INVENTION

The present invention provides a solution for the treatment or prevention of conditions involving muscular pain accompanied with said muscular imbalances, such as pain accompanying muscle contracture, pain accompanying muscle overload following exercise, muscle pain during episodes of fibromyalgia and, in particular, temporomandibular joint disorder, which is based on the synergistic combination of ibuprofen, its enantiomers or mixtures thereof, or a pharmaceutically acceptable salt thereof, a Mg²⁺ source, and ascorbic acid or a stereoisomer or a pharmaceutically acceptable salt thereof. This combination is particularly useful for the treatment or prevention of TMJD.

In a first aspect, the present invention is thus directed to a combination comprising:

a) ibuprofen, its enantiomers or mixtures thereof, or a pharmaceutically acceptable salt thereof;

b) a Mg²⁺ source; and

c) ascorbic acid or a stereoisomer or a pharmaceutically acceptable salt thereof.

The amount by weight of ibuprofen to the amount by weight of the Mg²⁺ source (expressed as Mg²⁺) is in the range from 2:1 to 6:1, and the amount by weight of ascorbic acid or the stereoisomer or the pharmaceutically acceptable salt thereof to the amount by weight of the Mg²⁺ source (expressed as Mg²⁺) is in the range from 1:1 to 12:1.

In a second aspect, the present invention relates to a combination as described in the first aspect for use as a medicament.

In a third aspect, the present invention relates to a combination as described in the first aspect for use in the treatment and/or prevention of a disease or condition involving muscular pain, optionally accompanied with muscular imbalances, such as temporomandibular joint disorder, pain accompanying muscle contracture, pain accompanying muscle overload following exercise and/or muscle pain during episodes of fibromyalgia.

In a fourth aspect, the present invention relates to a pharmaceutical composition comprising a combination as described in the first aspect and a pharmaceutically acceptable excipient.

### DESCRIPTION OF THE FIGURES

FIGS. 1 and 2 show the results obtained in Examples 2 and 5, respectively, namely likert scores of the level of pain evaluation between treatment A (black squares or crosses) and treatment B (white squares or circles) of Examples 2 and 5 for the likert items: 1) general pain relief; 2) relief of localized pain; 3) pain relief in comparison with other pharmacological treatments used for the management of TMDJ before this study such as analgesics and muscular relaxants in combination with analgesics; 4) treatment efficacy; 5) treatment discomfort; and 6) relief onset.

FIG. 3 shows the results obtained in Example 8.

### DESCRIPTION OF THE INVENTION

### Combination Product

In the first aspect, the present invention is directed to a combination comprising:

a) ibuprofen, its enantiomers or mixtures thereof, or a pharmaceutically acceptable salt thereof;

b) a Mg²⁺ source; and

c) ascorbic acid or a stereoisomer or a pharmaceutically acceptable salt thereof.

The amount by weight of ibuprofen to the amount by weight of the Mg²⁺ source (expressed as Mg²⁺) is in the range from 2:1 to 6:1, preferably 2:1 to 8:1 (with 3:1 to 6:1 being particularly preferred, such as 4:1 to 5:1), and the amount by weight of ascorbic acid or the stereoisomer or the pharmaceutically acceptable salt thereof to the amount by weight of the Mg²⁺ source (expressed as Mg²⁺) is in the range from 1:1 to 12:1 (with 2:1 to 6:1 being particularly preferred, such as 2.5:1 to 4:1).

The amount by weight of ibuprofen to the amount by weight of ascorbic acid or the stereoisomer or the pharmaceutically acceptable salt thereof is preferably in the range of 0.2:1 to 5:1, preferably 0.5:1 to 3:1.

Unless otherwise stated, in the context of the present invention, the ratios and amounts of components of the combination described herein, if the COX inhibitor such as ibuprofen and/or ascorbic acid are in the form of a salt, the values of said ratios and amounts refer to the equivalent COX inhibitor (acid) (e.g. ibuprofen (acid)) and/or equivalent free ascorbic acid, i.e. without counting the salt forming counterion. The amount of magnesium source is expressed as the equivalent ratio or amount of Mg2+ cation. Also, if the pharmaceutically acceptable salt of COX inhibitor and/or ascorbic acid is a magnesium salt, as explained above, said Mg2+ counterion forming the salt is also considered a Mg2+ source. Thus, in addition to the corresponding equivalent free COX inhibitor and/or equivalent free ascorbic acid, said Mg2+ also has to be taken into account as Mg2+ source when calculating the ratios and amounts of components in the combinations described herein.

The term “combination” refers to a group of 1, 2 or 3 compositions wherein the above mentioned products a), b) and c) are distributed among the 1, 2 or 3 compositions and wherein each of the 1, 2 or 3 compositions comprises at least one of products a), b) and c). Examples of combinations are, among others:
a combination comprising a first composition comprising a), a second composition comprising b), and a third composition comprising c);
a combination comprising a first composition comprising a), and a second composition comprising b) and c);
a combination comprising a first composition comprising a) and b), and a second composition comprising b and c);
a combination comprising a first composition comprising a) and b), a second composition comprising b), and a third composition comprising c);
a combination comprising a first composition comprising a) and b), a second composition comprising b) and c), and a third composition comprising c);
a combination comprising a first composition comprising a) and b), a second composition comprising b) and c), and a third composition comprising a) and c); and
a combination comprising a composition comprising a), b) and c).

A combination comprising a composition comprising a), b) and c) is preferred. The combination of the present invention and particularly the composition comprising a), b) and c) contains no other active ingredients besides a), b) and c).

The term “pharmaceutically acceptable salt” refers to any salt, which, upon administration to the recipient is capable of providing (directly or indirectly) a compound as described herein. However, it will be appreciated that non-pharmaceutically acceptable salts also fall within the scope of the invention since those may be useful in the preparation of pharmaceutically acceptable salts. The preparation of salts can be carried out by methods known in the art. For instance, pharmaceutically acceptable salts of compounds provided herein may be acid addition salts, base addition salts or metallic salts, and they can be synthesized from the parent compound which contains a basic or acidic moiety by conventional chemical methods. Generally, such salts are, for example, prepared by reacting the free acid or base forms of these compounds with a stoichiometric amount of the appropriate base or acid in water or in an organic solvent or in a mixture of the two. Generally, non-aqueous media like ether, ethyl acetate, ethanol, isopropanol or acetonitrile are preferred. Examples of the acid addition salts include mineral acid addition salts such as, for example, hydrochloride, hydrobromide, hydroiodide, sulphate, nitrate, phosphate, and organic acid addition salts such as, for example, acetate, maleate, fumarate, citrate, oxalate, succinate, tartrate, malate, mandelate, methanesulphonate, and p-toluenesulphonate. Examples of the alkali addition salts include inorganic salts such as, for example, ammonium, and organic alkali salts such as, for example, ethylenediamine, ethanolamine, N,N-dialkylenethanolamine, triethanolamine, glucamine and basic amino acids salts such as arginine and lysine. Examples of the metallic salts include, for example, sodium, potassium, calcium, magnesium, aluminium and lithium salts.

If the pharmaceutically acceptable salt is a magnesium salt, said Mg²⁺ counterion forming the salt is also considered as a Mg²⁺ source.

The term “stereoisomer” refers to compounds made up of the same atoms bonded by the same sequence of bonds but having different three-dimensional structures which are not interchangeable. Thus any given compound referred to herein is intended to represent any one of a racemate, one or more enantiomeric forms, one or more diastereomeric forms, one or more atropisomeric forms, and mixtures thereof.

The term “COX” or “cyclooxygenase” refers to a prostaglandin-endoperoxide synthase enzyme that is responsible for formation of prostanoids, including prostaglandins, prostacyclin and thromboxane. The resulting inhibition of prostaglandin and thromboxane synthesis has the effect of reduced inflammation, as well as antipyretic, antithrombotic and analgesic effects. Thus, pharmacological inhibition of COX can provide relief from the symptoms of inflammation and pain. COX-1 isoenzyme is an ubiquitously and constitutively expressed enzyme that is associated with the endoplasmic reticulum (ER). It is responsible for maintaining normal physiologic functions. It is normally expressed in the gastrointestinal tract, kidneys, and platelets and appears to be responsible for mediating the production of thromboxane A2 and prostaglandins. COX-2 isoenzyme is an inducible enzyme and is generally present at very low levels. It is mainly associated with the nuclear envelope and is primarily associated with inflammation. Cytokines and growth factors increase the expression of COX-2, mainly at inflammatory sites, producing prostaglandins, which mediate inflammation, pain, and fever.

In the context of the present invention, the term “COX inhibitors” refers to both COX-1 and COX-2 inhibitors. COX inhibitors are well known in the art. Examples of COX inhibitors are the non-selective COX-1 and COX-2 inhibitors, such as ibuprofen, meloxicam, aspirin, diflunisal, salsalate, diclofenac, naproxen, indomethacin, tolmetin, dexibuprofen, fenoprofen, mefenamic acid, ketoprofen, dexketoprofen, flurbiprofen, loxoprofen, oxaprozin, ketorolac, sulindac, etodolac, diclofenac, tenoxicam, piroxicam, paracetamol, ampiroxicam, bufexamac, zaltoprofen, flunixin meglumine, licofelone, lornoxicam, tolfenamic acid, acemetacin, triflusal, and pharmaceutically acceptable salts thereof; and the selective COX-2 inhibitors are nimesulide, celecoxib, parecoxib, rofecoxib, valdecoxib, lumiracoxib, ceracoxib, etoricoxib, aceclofenac, their enantiomers, pharmaceutically acceptable salts and mixtures thereof.

“Ibuprofen” refers to the racemic mixture of the R and S enantiomers of 2-(4-(2-methylpropyl)phenyl)propanoic acid, i.e. in a ratio of 1:1. Thus, “ibuprofen, its enantiomers or mixtures thereof” refers to the S enantiomer, the R enantiomer, and the mixture of the R and S enantiomers in any ratio including the racemic mixture. Preferred pharmaceutically acceptable salts of ibuprofen, its enantiomers or mixtures thereof are lysinate and arginate salts, more preferably a lysinate salt (ibuprofen lysinate).

The term “Mg²⁺ source” refers to any compound that comprises a Mg²⁺ moiety. Generally said Mg²⁺ source is a salt of an organic or inorganic compound, wherein the Mg²⁺ moiety is ionically bonded to the counter organic or inorganic anion. Examples of inorganic magnesium salts are magnesium sulfate, magnesium carbonate, magnesium chloride, magnesium oxide, magnesium hydroxide, magnesium bromide, magnesium fluoride, magnesium phosphate. Examples of organic magnesium salts are magnesium lactate (including magnesium DL-lactate, magnesium L-lactate, magnesium D-lactate), magnesium acetate, magnesium glycerophosphate, magnesium citrate, magnesium fumarate, magnesium gluconate, magnesium glycinate, magnesium aspartate (including magnesium DL-aspartate, magnesium L-aspartate, and magnesium D-aspartate), magnesium salicylate, magnesium ascorbate, magnesium gluceptate, magnesium pidolate. The Mg²⁺ source in the combination according to the present invention may include one or more compounds that comprise a Mg²⁺ moiety, preferably 1, 2, 3, 4, 5 or 6 compounds that comprise a Mg²⁺ moiety, even more preferably 1, 2 or 3 compounds that comprise a Mg²⁺ moiety.

The term “ascorbic acid” refers to any of the forms of Vitamin C, i.e. the enol and keto tautomers depicted below in Scheme 1, as well as the oxidized form dehydroascorbic acid.

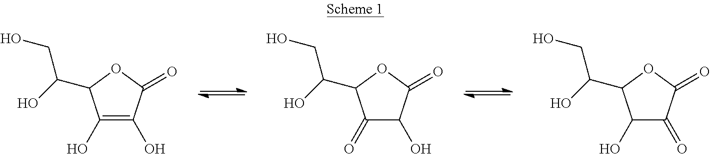 [SMILES: O=C1OC(C(O)CO)C(O)=C1O ; O=C1OC(C(O)CO)C(=O)C1O ; O=C1OC(C(O)CO)C(O)C1=O]

Stereoisomers of ascorbic acid may be selected from D- and L-ascorbic acid and mixtures thereof; preferably, the steroisomer of ascorbic acid is L-ascorbic acid.

In a preferred embodiment, the combination according to the present invention comprises:

a) ibuprofen, its enantiomers or mixtures thereof, or a pharmaceutically acceptable salt thereof;

b) a Mg²⁺ source; and

c) ascorbic acid or a stereoisomer or a pharmaceutically acceptable salt thereof;

with the proviso that when the Mg²⁺ source is selected from the group consisting of magnesium stearate, magnesium trisilicate, magnesium carbonate, magnesium oxide and magnesium hydroxide, then the combination comprises a further Mg²⁺ source which is not selected from the group consisting of magnesium stearate, magnesium trisilicate, magnesium carbonate, magnesium oxide and magnesium hydroxide.

In another preferred embodiment, the combination according to the present invention comprises an organic magnesium source; preferably, the organic magnesium salt is selected from the group consisting of magnesium gluconate, magnesium aspartate or a hydrate thereof (preferably a dihydrate), magnesium lactate (including magnesium DL-lactate, magnesium L-lactate, magnesium D-lactate), magnesium acetate, and magnesium glycerophosphate; more preferably, the organic magnesium source is magnesium aspartate or a hydrate thereof (preferably a dehydrate) or magnesium lactate (including magnesium DL-lactate, magnesium L-lactate, magnesium D-lactate); still more preferably, the organic magnesium source is magnesium L-lactate. It is also preferred to use magnesium gluconate in combination with magnesium oxide.

In another preferred embodiment, the combination according to the present invention comprises L-ascorbic acid or a pharmaceutically acceptable salt thereof.

In another preferred embodiment, the combination according to the present invention comprises ibuprofen, its enantiomers or mixtures thereof, a pharmaceutically acceptable salt thereof (such as the lysinate or arginate salt), magnesium lactate or magnesium aspartate and ascorbic acid or a pharmaceutically acceptable salt thereof.

The amount by weight of the COX inhibitor (i.e. ibuprofen) to the amount by weight of the Mg²⁺ source is in the range of 2:1 to 6:1, preferred being 4:1 to 6:1 such as 5:1.

The amount by weight of ascorbic acid or the stereoisomer or the pharmaceutically acceptable salt thereof to the amount by weight of the Mg²⁺ source is in the range from 1:1 to 12:1, preferably in the range of 2:1 to 5:1 or 6:1, more preferably 2.2:1 to 4.5:1, such as 4:1.

The amount by weight of ibuprofen to the amount by weight of ascorbic acid or the stereoisomer or the pharmaceutically acceptable salt thereof is preferably in the range of 0.2:1 to 5:1, preferably 0.5:1 to 3:1.

In a preferred embodiment, in the combination according to the present invention the amount by weight of ibuprofen to the amount by weight of the Mg²⁺ source is in the range from 2:1 to 6:1, and the amount by weight of ascorbic acid or the stereoisomer or the pharmaceutically acceptable salt thereof to the amount by weight of the Mg²⁺ source is in the range from 1:1 to 12:1; preferably the amount by weight of the COX inhibitor to the amount by weight of the Mg²⁺ source is in the range from 2:1 to 6:1, and the amount by weight of ascorbic acid or the stereoisomer or the pharmaceutically acceptable salt thereof to the amount by weight of the Mg²⁺ source is in the range from 2:1 to 5:1.

In a particular embodiment, the combination according to the present invention comprises ibuprofen, its enantiomers or mixtures thereof, or pharmaceutically acceptable salts thereof, a Mg²⁺ source which is an organic magnesium salt, L-ascorbic acid or a pharmaceutically acceptable salt thereof, wherein the amount by weight of ibuprofen to the amount by weight of the Mg²⁺ source is in the range from 2:1 to 6:1, and the amount by weight of L-ascorbic acid or the pharmaceutically acceptable salt thereof to the amount by weight of the Mg²⁺ source is in the range from 1:1 to 12:1; preferably the amount by weight of ibuprofen to the amount by weight of the Mg²⁺ source is in the range from 2:1 to 6:1, and the amount by weight of L-ascorbic acid or the pharmaceutically acceptable salt thereof to the amount by weight of the Mg²⁺ source is in the range from 2:1 to 5:1.

In another particular embodiment, the combination according to the present invention comprises ibuprofen, its enantiomers or mixtures thereof, a Mg²⁺ source which is an organic magnesium salt selected from the group consisting of magnesium lactate, magnesium acetate, and magnesium glycerophosphate, L-ascorbic acid or a pharmaceutically acceptable salt thereof, wherein the amount by weight of the COX inhibitor to the amount by weight of the Mg²⁺ source is in the range from 2:1 to 6:1 times the weight of the Mg²⁺ source, and the amount by weight of L-ascorbic acid or the pharmaceutically acceptable salt thereof to the amount by weight of the Mg²⁺ source is in the range from 1:1 to 12:1; preferably the amount by weight of ibuprofen to the amount by weight of the Mg²⁺ source is in the range from 3:1 to 6:1 times the weight of the Mg²⁺ source, and the amount by weight of L-ascorbic acid or the pharmaceutically acceptable salt thereof to the amount by weight of the Mg²⁺ source is in the range from 2:1 to 5:1.

In another preferred embodiment, the amount by weight of ibuprofen, its enantiomers or mixtures thereof, a hydrate or a pharmaceutically acceptable salt thereof, expressed as equivalent ibuprofen acid, to the amount by weight of the Mg2+ source expressed as Mg2+ is in the range from 2:1 to 6:1 (preferably 4:1 to 6:1 such as 5:1) and/or the amount by weight of ascorbic acid or the stereoisomer, the hydrate or the pharmaceutically acceptable salt thereof, expressed as equivalent free ascorbic acid, to the amount by weight of the Mg2+ source expressed as Mg2+ is in the range from 1:1 to 8:1 (preferably 2:1 to 6:1 such as 4:1). Preferably, the amount by weight of ascorbic acid or the stereoisomer, the hydrate or the pharmaceutically acceptable salt thereof, expressed as equivalent free ascorbic acid, to the amount by weight of the Mg2+ source expressed as Mg2+ is in the range from 2:1 to 5:1. More preferably, the amount by weight of ibuprofen, its enantiomers or mixtures thereof, a hydrate or a pharmaceutically acceptable salt thereof, expressed as equivalent ibuprofen acid, to the amount by weight of the Mg2+ source expressed as Mg2+ is in the range from 2:1 to 6:1 and/or the amount by weight of ascorbic acid or the stereoisomer, the hydrate or the pharmaceutically acceptable salt thereof, expressed as equivalent free ascorbic acid, to the amount by weight of the Mg2+ source expressed as Mg2+ is in the range from 2.2:1 to 4.5:1. More preferably, the amount by weight of ascorbic acid or the stereoisomer, the hydrate or the pharmaceutically acceptable salt thereof, expressed as equivalent free ascorbic acid, to the amount by weight of the Mg2+ source expressed as Mg2+ is in the range from 1:1 to 5:1. More preferably, the amount by weight of ibuprofen, its enantiomers or mixtures thereof, a hydrate or a pharmaceutically acceptable salt thereof, expressed as equivalent ibuprofen acid, to the amount by weight of the Mg2+ source expressed as Mg2+ is in the range from 3:1 to 5.5:1 and/or the amount by weight of ascorbic acid or the stereoisomer, the hydrate or the pharmaceutically acceptable salt thereof, expressed as equivalent free ascorbic acid, to the amount by weight of the Mg2+ source expressed as Mg2+ is in the range from 2:1 to 5:1. Still more preferably, the amount by weight of ibuprofen, its enantiomers or mixtures thereof, a hydrate or a pharmaceutically acceptable salt thereof, expressed as equivalent ibuprofen acid, to the amount by weight of the Mg2+ source expressed as Mg2+ is in the range from 3:1 to 5.5:1 and/or the amount by weight of ascorbic acid or the stereoisomer, the hydrate or the pharmaceutically acceptable salt thereof, expressed as equivalent free ascorbic acid, to the amount by weight of the Mg2+ source expressed as Mg2+ is in the range from 2.2:1 to 4.5:1.

In another particular embodiment, the combination according to the present invention comprises ibuprofen, its enantiomers or mixtures thereof, or a pharmaceutically acceptable salt thereof (such as the lysinate or arginate salt), magnesium lactate, preferably L-magnesium lactate, and L-ascorbic acid or a pharmaceutically acceptable salt thereof, wherein the amount by weight of ibuprofen, its enantiomers or mixtures thereof, or a pharmaceutically acceptable salt thereof (such as the lysinate or arginate salt) to the amount by weight of magnesium lactate, preferably L-magnesium lactate, or magnesium aspartate is in the range from 2:1 to 6:1, and the amount by weight of L-ascorbic acid or the pharmaceutically acceptable salt thereof to the amount by weight of magnesium lactate, preferably L-magnesium lactate, or magnesium aspartate is in the range from 1:1 to 12:1; preferably the amount by weight of ibuprofen, its enantiomers or mixtures thereof, or a pharmaceutically acceptable salt thereof (such as the lysinate or arginate salt) to the amount by weight of magnesium lactate, preferably L-magnesium lactate, or magnesium aspartate is in the range from 3:1 to 6:1, and the amount by weight of L-ascorbic acid or the pharmaceutically acceptable salt thereof to the amount by weight of magnesium lactate, preferably L-magnesium lactate, or magnesium aspartate is in the range from 2:1 to 5:1.

In a particular embodiment, the combination according to the present invention comprises:
from 15 mg to 1500 mg of ibuprofen, and preferably 300 to 700 mg of ibuprofen,
from 50 mg to 150 mg of a Mg²⁺ source, and
from 200 mg to 600 mg of ascorbic acid or a stereoisomer or a pharmaceutically acceptable salt thereof.

The above combination is preferably used for administration once a day.

In a particular embodiment, the combination according to the present invention comprises:
from 15 mg to 1500 mg of ibuprofen, its enantiomers or mixtures thereof, or pharmaceutically acceptable salts thereof,
from 200 mg to 350 mg of a Mg²⁺ source which is an organic magnesium salt, and
from 500 mg to 2000 mg of L-ascorbic acid or a pharmaceutically acceptable salt thereof.

The above combination is preferably used for administration once a day.

In another particular embodiment, the combination according to the present invention comprises:
from 15 mg to 1500 mg of ibuprofen, its enantiomers, or mixtures thereof, or pharmaceutically acceptable salts thereof,
from 200 mg to 350 mg of a Mg²⁺ source which is an organic magnesium salt selected from the group consisting of magnesium lactate, magnesium acetate, and magnesium glycerophosphate, and
from 500 mg to 2000 mg of L-ascorbic acid or a pharmaceutically acceptable salt thereof.

The above combination is preferably used for administration once a day.

In any of the combinations defined above, which are preferably used for administration once a day, ibuprofen, its enantiomers or mixtures thereof, or a pharmaceutically acceptable salt thereof in an amount (expressed as free ibuprofen) of from 1000 mg to 1500 mg is particularly preferred.

In another particular embodiment, the combination according to the present invention comprises:
from 1000 mg to 1500 mg of ibuprofen, its enantiomers or mixtures thereof, or a pharmaceutically acceptable salt thereof (such as the lysinate or arginate salt),
from 200 mg to 350 mg of magnesium lactate, preferably magnesium L-lactate, and
from 500 mg to 2000 mg of L-ascorbic acid or a pharmaceutically acceptable salt thereof.

The above combination is preferably used for administration once a day.

In another particular embodiment, the combination according to the present invention comprises:
from 7.5 mg to 750 mg of ibuprofen,
from 100 mg to 175 mg of a Mg²⁺ source, and
from 250 mg to 1000 mg of ascorbic acid or a stereoisomer or a pharmaceutically acceptable salt thereof.

The above combination is preferably used for administration twice a day.

In a particular embodiment, the combination according to the present invention comprises:
from 7.5 mg to 750 mg of ibuprofen, its enantiomers or mixtures thereof, or pharmaceutically acceptable salts thereof,
from 100 mg to 175 mg of a Mg²⁺ source which is an organic magnesium salt, and
from 250 mg to 1000 mg of L-ascorbic acid or a pharmaceutically acceptable salt thereof.

The above combination is preferably used for administration twice a day.

In another particular embodiment, the combination according to the present invention comprises:
from 7.5 mg to 750 mg of ibuprofen, its enantiomers or mixtures thereof, or a pharmaceutically acceptable salt thereof,
from 100 mg to 175 mg of a Mg²⁺ source which is an organic magnesium salt selected from the group consisting of magnesium lactate, magnesium acetate, and magnesium glycerophosphate, and
from 250 mg to 1000 mg of L-ascorbic acid or a pharmaceutically acceptable salt thereof.

The above combination is preferably used for administration twice a day.

In any of the combinations defined above, which are preferably used for administration twice a day, ibuprofen, its enantiomers or mixtures thereof, or a pharmaceutically acceptable salt thereof in an amount (expressed as free ibuprofen) of from 500 mg to 750 mg is particularly preferred.

In another particular embodiment, the combination according to the present invention comprises:
from 500 mg to 750 mg of a ibuprofen, its enantiomers or mixtures thereof, or a pharmaceutically acceptable salt thereof (such as the lysinate or arginate salt),
from 100 mg to 175 mg of magnesium lactate, preferably magnesium L-lactate, and
from 250 mg to 1000 mg of L-ascorbic acid or a pharmaceutically acceptable salt thereof.

The above combination is preferably used for administration twice a day.

In a preferred embodiment, the combination according to the present invention comprises:
from 5 mg to 600 mg of ibuprofen,
from 60 mg to 150 mg of a Mg²⁺ source, and
from 200 mg to 600 mg of ascorbic acid or a stereoisomer or a pharmaceutically acceptable salt thereof.

The above combination is preferably used for administration three times a day.

In a particular embodiment, the combination according to the present invention comprises:
from 5 mg to 500 mg of ibuprofen, its enantiomers or mixtures thereof, or pharmaceutically acceptable salts thereof,
from 60 mg to 150 mg of a Mg²⁺ source which is an organic magnesium salt, and
from 200 mg to 600 mg of L-ascorbic acid or a pharmaceutically acceptable salt thereof.

The above combination is preferably used for administration three times a day.

In another particular embodiment, the combination according to the present invention comprises:
from 5 mg to 600 mg of ibuprofen, its enantiomers or mixtures thereof, and meloxicam, or a pharmaceutically acceptable salt thereof,
from 60 mg to 150 mg of a Mg²⁺ source which is an organic magnesium salt selected from the group consisting of magnesium lactate, magnesium acetate, and magnesium glycerophosphate, and
from 200 mg to 600 mg of L-ascorbic acid or a pharmaceutically acceptable salt thereof.

The above combination is preferably used for administration three times a day.

In any of the combinations defined above, which are preferably used for administration three times a day, it is particularly preferred that ibuprofen, its enantiomers or mixtures thereof, or a pharmaceutically acceptable salt thereof is present in an amount (expressed as free ibuprofen) of from 300 mg to 500 mg.

In another particular embodiment, the combination according to the present invention comprises:
from 300 mg to 600 mg (preferably 400 to 500 mg) of a ibuprofen, its enantiomers or mixtures thereof, or a pharmaceutically acceptable salt thereof (such as the lysinate or arginate salt),
from 60 mg to 150 mg of magnesium, such as magnesium lactate, preferably magnesium L-lactate, and
from 200 mg to 600 mg of L-ascorbic acid or a pharmaceutically acceptable salt thereof.

The above combination is preferably used for administration three times a day.

In the ratios and amounts of components of the combination defined above, when ibuprofen and/or the ascorbic acid are in the form of a salt, the values of said ratios and amounts refer to the equivalent free COX inhibitor and/or equivalent free ascorbic acid, i.e. without counting the salt forming counterion.

If the pharmaceutically acceptable salt of ibuprofen and/or ascorbic acid is a magnesium salt, as explained above, said Mg²⁺ counterion forming the salt is also considered a Mg²⁺ source. Thus, in addition to the corresponding equivalent free ibuprofen and/or equivalent free ascorbic acid, said Mg²⁺ also has to be taken into account when calculating the above ratios and amounts of components in the compositions.

In a preferred embodiment, the components of the combination according to the present invention, i.e. ibuprofen, the Mg²⁺ source and the ascorbic acid or stereoisomer or pharmaceutically acceptable salt thereof, as defined above, form part of the same composition.

In an alternative embodiment, the components of the combination according to the present invention, i.e. ibuprofen, the Mg²⁺ source and the ascorbic acid or stereoisomer or pharmaceutically acceptable salt thereof, as defined above, form part of different compositions. For example, ibuprofen and the Mg²⁺ source, as defined above, form part of one composition and the ascorbic acid or stereoisomer or pharmaceutically acceptable salt thereof, as defined above, forms part of another composition; ibuprofen and the ascorbic acid or stereoisomer or pharmaceutically acceptable salt thereof, as defined above, form part of one composition, and the Mg²⁺ source forms part of another composition; the Mg²⁺ source and the ascorbic acid or stereoisomer or pharmaceutically acceptable salt thereof, as defined above, form part of one composition, and ibuprofen, as defined above, forms part of another composition; or ibuprofen, as defined above, forms part of a first composition, the Mg²⁺ source, as defined above, forms part of a second composition, and the ascorbic acid or stereoisomer or pharmaceutically acceptable salt thereof, as defined above, forms part of a third composition.

In a preferred embodiment, the combination according to the present invention comprises:
ibuprofen, its enantiomers or mixtures thereof, a hydrate or a pharmaceutically acceptable salt thereof (preferably ibuprofen lysinate or arginate, more preferably ibuprofen lysinate) in an amount of from 300 mg to 700 mg expressed as equivalent ibuprofen acid,
a Mg2+ source, in an amount of from 50 mg to 150 mg expressed as Mg2+, and
ascorbic acid or a stereoisomer, a hydrate or a pharmaceutically acceptable salt thereof, in an amount of from 200 mg to 600 mg expressed as equivalent free ascorbic acid.

The above combination is preferably used for administration three times a day.

In a particular embodiment, the combination according to the present invention comprises:
ibuprofen, its enantiomers or mixtures thereof, a hydrate or a pharmaceutically acceptable salt thereof (preferably ibuprofen lysinate or arginate, more preferably ibuprofen lysinate) in an amount of from 350 mg to 450 mg expressed as equivalent ibuprofen acid,
a Mg2+ source, in an amount of from 50 mg to 150 mg expressed as Mg2+, and
ascorbic acid or a stereoisomer, a hydrate or a pharmaceutically acceptable salt thereof in an amount of from 200 mg to 400 mg expressed as equivalent free ascorbic acid.

The above combination is preferably used for administration three times a day.

In a particular embodiment, the combination according to the present invention comprises:
ibuprofen, its enantiomers or mixtures thereof, a hydrate or a pharmaceutically acceptable salt thereof (preferably ibuprofen lysinate or arginate, more preferably ibuprofen lysinate) in an amount of from 300 mg to 700 mg expressed as equivalent ibuprofen acid,
a Mg2+ source selected from magnesium aspartate or a hydrate thereof (preferably a dihydrate) or a combination of magnesium oxide and magnesium gluconate or hydrates thereof, in an amount of from 50 mg to 150 mg expressed as Mg2+, and
L-ascorbic acid, a hydrate or a pharmaceutically acceptable salt thereof in an amount of from 200 mg to 600 mg expressed as equivalent free ascorbic acid.

The above combination is preferably used for administration three times a day.

In a particular embodiment, the combination according to the present invention comprises:
ibuprofen, its enantiomers or mixtures thereof, a hydrate or a pharmaceutically acceptable salt thereof (preferably ibuprofen lysinate or arginate, more preferably ibuprofen lysinate) in an amount of from 350 mg to 450 mg expressed as equivalent ibuprofen acid,
a Mg2+ source selected from magnesium aspartate or a hydrate thereof (preferably a dihydrate) or a combination of magnesium oxide and magnesium gluconate or hydrates thereof in an amount of from 50 mg to 150 mg expressed as Mg2+, and
L-ascorbic acid, a hydrate or a pharmaceutically acceptable salt thereof in an amount of from 200 mg to 400 mg expressed as equivalent free ascorbic acid.

The above combination is preferably used for administration three times a day.

In a particular embodiment, the combination according to the present invention comprises:
ibuprofen lysinate (from 598 mg to 769 mg of ibuprofen lysinate) or a hydrate thereof equivalent to an amount of from 350 mg to 450 mg of ibuprofen acid,
magnesium aspartate (from 593 mg to 1781 mg of magnesium aspartate) or a hydrate thereof equivalent to an amount of from 50 mg to 150 mg of Mg2+, preferably magnesium aspartate dihydrate, and
from 200 mg to 400 mg of L-ascorbic acid or a hydrate thereof.

The above combination is preferably used for administration three times a day.

In a preferred embodiment, the combination according to the present invention comprises:
ibuprofen lysinate (from 598 mg to 769 mg of ibuprofen lysinate) or a hydrate thereof equivalent to an amount of from 350 mg to 450 mg of ibuprofen acid,
magnesium oxide and magnesium gluconate or hydrates thereof equivalent to an amount of from 50 mg to 150 mg of Mg2+, and
from 200 mg to 400 mg of L-ascorbic acid or a hydrate thereof.

The above combination is preferably used for administration three times a day.

In a preferred embodiment, the combination according to the present invention comprises:
ibuprofen lysinate (from 940 mg to 1111 mg of ibuprofen lysinate) or a hydrate thereof equivalent to an amount of from 550 mg to 650 mg of ibuprofen acid,
magnesium oxide and magnesium gluconate or hydrates thereof equivalent to an amount of from 90 mg to 150 mg of Mg2+, and
from 200 mg to 400 mg of L-ascorbic acid or a hydrate thereof.

The above combination is preferably used for administration three times a day.

In a particular embodiment, the combination according to the present invention comprises:
ibuprofen, its enantiomers or mixtures thereof, a hydrate or a pharmaceutically acceptable salt thereof (preferably ibuprofen lysinate or arginate, more preferably ibuprofen lysinate) in an amount of from 450 mg to 1050 mg expressed as equivalent ibuprofen acid,
a Mg2+ source in an amount of from 75 mg to 225 mg expressed as Mg2+, and
ascorbic acid or a stereoisomer, a hydrate or a pharmaceutically acceptable salt thereof in an amount of from 300 mg to 900 mg expressed as equivalent free ascorbic acid.

The above combination is preferably used for administration twice a day.

In a particular embodiment, the combination according to the present invention comprises:
ibuprofen, its enantiomers or mixtures thereof, a hydrate or a pharmaceutically acceptable salt thereof (preferably ibuprofen lysinate or arginate, more preferably ibuprofen lysinate) in an amount of from 525 mg to 675 mg expressed as equivalent ibuprofen acid,
a Mg2+ source in an amount of from 75 mg to 225 mg expressed as Mg2+, and
ascorbic acid or a stereoisomer, a hydrate or a pharmaceutically acceptable salt thereof in an amount of from 300 mg to 600 mg expressed as equivalent free ascorbic acid.

The above combination is preferably used for administration twice a day.

In a particular embodiment, the combination according to the present invention comprises:
ibuprofen, its enantiomers or mixtures thereof, a hydrate or a pharmaceutically acceptable salt thereof (preferably ibuprofen lysinate or arginate, more preferably ibuprofen lysinate) in an amount of from 450 mg to 1050 mg expressed as equivalent ibuprofen acid,
a Mg2+ source selected from magnesium aspartate or a hydrate thereof (preferably magnesium aspartate dihydrate) or a combination of magnesium oxide and magnesium gluconate in an amount of from 75 mg to 225 mg expressed as Mg2+, and
L-ascorbic acid, a hydrate or a pharmaceutically acceptable salt thereof in an amount of from 300 mg to 900 mg expressed as equivalent free ascorbic acid.

The above combination is preferably used for administration twice a day.

In a particular embodiment, the combination according to the present invention comprises:
ibuprofen, its enantiomers or mixtures thereof, a hydrate or a pharmaceutically acceptable salt thereof (preferably ibuprofen lysinate or arginate, more preferably ibuprofen lysinate) in an amount of from 525 mg to 675 mg expressed as equivalent ibuprofen acid,
a Mg2+ source selected from magnesium aspartate or a hydrate thereof (preferably magnesium aspartate dihydrate) or a combination of magnesium oxide and magnesium gluconate in an amount of from 75 mg to 225 mg expressed as Mg2+, and
L-ascorbic acid, a hydrate or a pharmaceutically acceptable salt thereof in an amount of from 300 mg to 600 mg expressed as equivalent free ascorbic acid.

The above combination is preferably used for administration twice a day.

In a particular embodiment, the combination according to the present invention comprises:
ibuprofen, its enantiomers or mixtures thereof, a hydrate or a pharmaceutically acceptable salt thereof (preferably ibuprofen lysinate or arginate, more preferably ibuprofen lysinate) in an amount of from 900 mg to 2100 mg expressed as equivalent ibuprofen acid,
a Mg2+ source in an amount of from 150 mg to 450 mg expressed as Mg2+, and
ascorbic acid or a stereoisomer, a hydrate or a pharmaceutically acceptable salt thereof in an amount of from 600 mg to 1800 mg expressed as equivalent free ascorbic acid.

The above combination is preferably used for administration once a day.

In a particular embodiment, the combination according to the present invention comprises:
ibuprofen, its enantiomers or mixtures thereof, a hydrate or a pharmaceutically acceptable salt thereof (preferably ibuprofen lysinate or arginate, more preferably ibuprofen lysinate) in an amount of from 1050 mg to 1350 mg expressed as equivalent ibuprofen acid,
a Mg2+ source in an amount of from 150 mg to 450 mg expressed as Mg2+, and
ascorbic acid or a stereoisomer, a hydrate or a pharmaceutically acceptable salt thereof in an amount of from 600 mg to 1200 mg expressed as equivalent free ascorbic acid.

The above combination is preferably used for administration once a day.

In a particular embodiment, the combination according to the present invention comprises:
ibuprofen, its enantiomers or mixtures thereof, a hydrate or a pharmaceutically acceptable salt thereof (preferably ibuprofen lysinate or arginate, more preferably ibuprofen lysinate) in an amount of from 900 mg to 2100 mg expressed as equivalent ibuprofen acid,
a Mg2+ source selected from magnesium aspartate or a hydrate thereof (preferably magnesium aspartate dihydrate) or a combination of magnesium oxide and magnesium gluconate or hydrates thereof in an amount of from 150 mg to 450 mg expressed as Mg2+, and
L-ascorbic acid, a hydrate or a pharmaceutically acceptable salt thereof in an amount of from 600 mg to 1800 mg expressed as equivalent free ascorbic acid.

The above combination is preferably used for administration once a day.

In a particular embodiment, the combination according to the present invention comprises:
ibuprofen, its enantiomers or mixtures thereof, a hydrate or a pharmaceutically acceptable salt thereof (preferably ibuprofen lysinate or arignate, more preferably ibuprofen lysinate) in an amount of from 1050 mg to 1350 mg expressed as equivalent ibuprofen acid,
a Mg2+ source selected from magnesium aspartate or a hydrate thereof (preferably magnesium aspartate dihydrate) or a combination of magnesium oxide and magnesium gluconate or hydrates thereof in an amount of from 150 mg to 450 mg expressed as Mg2+, and
L-ascorbic acid, a hydrate or a pharmaceutically acceptable salt thereof in an amount of from 600 mg to 1200 mg expressed as equivalent free ascorbic acid.

The above combination is preferably used for administration once a day.

In a preferred embodiment, the components of the combination according to the present invention, i.e. ibuprofen, its enantiomers or mixtures thereof, a hydrate or a pharmaceutically acceptable salt thereof, the Mg2+ source and the ascorbic acid or stereoisomer, hydrate or pharmaceutically acceptable salt thereof, as defined above, form part of the same composition.

In an alternative embodiment, the components of the combination according to the present invention, i.e. ibuprofen, its enantiomers or mixtures thereof, a hydrate or a pharmaceutically acceptable salt thereof, the Mg2+ source and the ascorbic acid or stereoisomer, hydrate or pharmaceutically acceptable salt thereof, as defined above, form part of different compositions. For example, ibuprofen, its enantiomers or mixtures thereof, a hydrate or a pharmaceutically acceptable salt thereof, and the Mg2+ source, as defined above, form part of one composition and the ascorbic acid or stereoisomer, hydrate or pharmaceutically acceptable salt thereof, as defined above, forms part of another composition; ibuprofen, its enantiomers or mixtures thereof, a hydrate or a pharmaceutically acceptable salt thereof, and the ascorbic acid or stereoisomer, hydrate or pharmaceutically acceptable salt thereof, as defined above, form part of one composition, and the Mg2+ source forms part of another composition; the Mg2+ source and the ascorbic acid or stereoisomer, hydrate or pharmaceutically acceptable salt thereof, as defined above, form part of one composition, and ibuprofen, its enantiomers or mixtures thereof, a hydrate or a pharmaceutically acceptable salt thereof, as defined above, forms part of another composition; or ibuprofen, its enantiomers or mixtures thereof, a hydrate or a pharmaceutically acceptable salt thereof, as defined above, forms part of a first composition, the Mg2+ source, as defined above, forms part of a second composition, and the ascorbic acid or stereoisomer, hydrate or pharmaceutically acceptable salt thereof, as defined above, forms part of a third composition.

### Medical Uses

The combination of a COX inhibitor, a Mg²⁺ source, and ascorbic acid or a stereoisomer or a pharmaceutically acceptable salt thereof, as described above, provides a synergistic effect when used in the treatment and/or prevention of conditions involving muscular pain, optionally accompanied with muscular imbalances, such as temporomandibular joint disorder, pain accompanying muscle contracture, pain accompanying muscle overload following exercise and/or muscle pain during episodes of fibromyalgia.

Thus, in a second aspect the present invention relates to a combination comprising ibuprofen, a Mg²⁺ source, and ascorbic acid or a stereoisomer or a pharmaceutically acceptable salt thereof, as described above, for use of a medicament.

Another aspect of the present invention relates to the use of a combination comprising ibuprofen, a Mg²⁺ source, and ascorbic acid or a stereoisomer or a pharmaceutically acceptable salt thereof, as defined above, for the manufacture of a medicament.

In a further aspect, the present invention relates to a combination comprising ibuprofen, a Mg²⁺ source, and ascorbic acid or a stereoisomer or a pharmaceutically acceptable salt thereof, as defined above, for use in the treatment and/or prevention of conditions involving muscular pain, optionally accompanied with muscular imbalances, such as temporomandibular joint disorder, pain accompanying muscle contracture, pain accompanying muscle overload following exercise and/or muscle pain during episodes of fibromyalgia; preferably TMDJ.

In the context of the present invention the term “muscular imbalances” is used to designate, among others, muscle contractures, muscle overload, muscle micro-tears, and muscle cramps.

Another aspect of the present invention relates to the use of a combination comprising ibuprofen, a Mg²⁺ source, and ascorbic acid or a stereoisomer or a pharmaceutically acceptable salt thereof, as defined above, in the manufacture of a medicament for the treatment and/or prevention of conditions involving muscular pain, optionally accompanied with muscular imbalances, such as temporomandibular joint disorder, pain accompanying muscle contracture, pain accompanying muscle overload following exercise and/or muscle pain during episodes of fibromyalgia; preferably TMDJ.

Another aspect relates to a method of treatment and/or prevention of conditions involving muscular pain, optionally accompanied with muscular imbalances, such as temporomandibular joint disorder, pain accompanying muscle contracture, pain accompanying muscle overload following exercise and/or muscle pain during episodes of fibromyalgia, preferably TMDJ, in a subject in need thereof, which comprises the administration of a therapeutically effective amount of a combination comprising ibuprofen or a pharmaceutically acceptable salt thereof, a Mg²⁺ source, and ascorbic acid or a stereoisomer or a pharmaceutically acceptable salt thereof, as defined above.

The terms “treating” and “treatment”, as used herein, mean reversing, alleviating, inhibiting the progress of, the disease or condition to which such term applies, or one or more symptoms of such disease or condition, such as muscular pain, muscle contractures, muscle overload, muscle micro-tears, and muscle cramps.

The temporomandibular joint is the joint of the jaw. There are two temporomandibular joints, one on each side, working in unison. The temporomandibular joint comprises an articular disc that is composed of fibrocartilagenous tissue which is positioned between the two bones that form the joint, the upper temporal bone and the lower jaw bone (mandibule). The disc divides each joint into two. The lower joint compartment formed by the mandibule and the articular disc is involved in rotational movement—this is the initial movement of the jaw when the mouth opens. The upper joint compartment formed by the articular disk and the temporal bone is involved in translational movement—this is the secondary gliding motion of the jaw as it is opened widely.

Temporomandibular joint disorder (TMJD) or temporomandibular joint syndrome is a disease that is caused by joint degeneration and/or dysfunction of the muscles of mastication, leading to joint inflammation or painful progressive chronic pain and reduced quality of life. TMJD is characterized by chronic or acute musculoskeletal pain with dysfunction of the masticatory system, such as restricted mandibular movement.

TMJD may arise due to stress, jaw malocclusion, jaw clenching which is often due to anxiety and psychosocial stress, bruxism (psychogenic grinding of the teeth), other musculoskeletal problems such as degenerative joint disease, internal joint derangements, cervical rotation, and dental manipulation, and rarely from trauma. TMJD is typically seen in young women, often arising in the third of fourth decade of life.

In particular, when referring to the treatment of TMJD, the terms “treating” and “treatment” mean reversing, alleviating, inhibition the progress of one or more of the following symptoms:
pain, including tenderness in the jaw, aching pain in or around the ear, aching facial pain or nearly constant pain, wherein said pain may be present whether the temporomandibular joint is moving or not;
difficulty in opening the mouth fully;
difficulty or discomfort while chewing;
a clicking or popping sensation in the joint;
locking of the joint that makes it hard to open or close the mouth;
headache;
uncomfortable bite; and/or
uneven bite because one or more teeth are making contact with each other before the other teeth do

The terms “preventing” and “prevention”, as used herein, means inhibiting the onset of the disease or condition to which this term applies, or one or more symptoms of such disease or condition.

By an “effective amount” or a “therapeutically effective amount” of a drug or pharmacologically active agent is meant a non-toxic, but sufficient amount of the drug or agent to provide the desired effect. In the combination therapy of the present invention, an “effective amount” of one component of the combination is the amount of that compound that is effective to provide the desired effect when used in combination with the other components of the combination. The amount that is “effective” will vary from subject to subject, depending on the age and general condition of the individual, the particular active agent or agents, and the like. Thus, it is not always possible to specify an exact “effective amount”. However, an appropriate “effective” amount in any individual case may be determined by one of ordinary skill in the art using routine experimentation.

ibuprofen, the Mg²⁺ source, and the ascorbic acid or stereoisomer or pharmaceutically acceptable salt thereof, as defined above, may be administered simultaneously, sequentially or separately. In any of these particular administration modes, the combination according to the present invention is designed to enable the simultaneous, sequential or separate administration of ibuprofen, the Mg²⁺ source, and the ascorbic acid or stereoisomer or pharmaceutically acceptable salt thereof. In a particular embodiment, ibuprofen, the Mg²⁺ source, and the ascorbic acid or stereoisomer or pharmaceutically acceptable salt thereof, as defined above, are administered simultaneously. In another particular embodiment, ibuprofen, the Mg²⁺ source, and the ascorbic acid or stereoisomer or pharmaceutically acceptable salt thereof, as defined above, are administered sequentially.

Simultaneous administration may, e.g., take place in the form of one composition comprising ibuprofen, the Mg²⁺ source, and the ascorbic acid or stereoisomer or pharmaceutically acceptable salt thereof, as defined above, or by simultaneously administering, i.e. administering at the same time, ibuprofen, the Mg²⁺ source, and the ascorbic acid or stereoisomer or pharmaceutically acceptable salt thereof which are formulated independently, i.e. not forming part of the same composition.

Sequential administration preferably means administration of ibuprofen, at one time point, the Mg²⁺ source at another time point, and the ascorbic acid or stereoisomer or pharmaceutically acceptable salt thereof at a different time point, in a chronically staggered manner; the administration of ibuprofen and the Mg²⁺ source at one time point, and the ascorbic acid or stereoisomer or pharmaceutically acceptable salt thereof at a different time point in a chronically staggered manner; the administration of ibuprofen and the ascorbic acid or stereoisomer or pharmaceutically acceptable salt thereof at one time point, and the Mg²⁺ source at different time point in a chronically staggered manner; or the administration of the Mg²⁺ source and the ascorbic acid or stereoisomer of pharmaceutically acceptable salt thereof at one time point, and ibuprofen at a different time point in a chronically staggered manner.

Separate administration preferably means administration of ibuprofen, at one time point, the Mg²⁺ source at another time point, and the ascorbic acid or stereoisomer or pharmaceutically acceptable salt thereof, as defined above, independently of each other at different time points; the administration of ibuprofen and the Mg²⁺ source at one time point, and the ascorbic acid or stereoisomer or pharmaceutically acceptable salt thereof at a different time point independently of the each other at different time points; the administration of ibuprofen and the ascorbic acid or stereoisomer or pharmaceutically acceptable salt thereof at one time point, and the Mg²⁺ source independently of each other at different time points; or the administration of the Mg²⁺ source and the ascorbic acid or stereoisomer of pharmaceutically acceptable salt thereof at one time point, and ibuprofen independently of each other at different time points.

When administered sequentially or separately, ibuprofen, the Mg²⁺ source and the ascorbic acid or stereoisomer or pharmaceutically acceptable salt thereof, as defined above, may be in any order. In one particular embodiment, ibuprofen is administered first. In another particular embodiment, the Mg²⁺ source is administered first. In another particular embodiment, the ascorbic acid or stereoisomer of pharmaceutically acceptable salt thereof is administered first. In another particular embodiment, ibuprofen or and the Mg²⁺ source are administered first. In another particular embodiment, ibuprofen and the ascorbic acid or stereoisomer or pharmaceutically acceptable salt thereof are administered first. In another particular embodiment, the Mg²⁺ source and the ascorbic acid or stereoisomer of pharmaceutically acceptable salt thereof are administered first.

In a particular embodiment the combination for use according to the present invention comprises ibuprofen, the Mg²⁺ source and the ascorbic acid or stereoisomer or pharmaceutically acceptable salt thereof, as defined above, forming part of the same composition for simultaneous administration.

In another particular embodiment, the combination for use according to the present invention comprises ibuprofen, the Mg²⁺ source and the ascorbic acid or stereoisomer or pharmaceutically acceptable salt thereof, as defined above, provided as separate compositions, preferably for simultaneous administration.

ibuprofen, the Mg²⁺ source and the ascorbic acid or stereoisomer or pharmaceutically acceptable salt thereof, as defined above may be administered at different dosages depending on different factors such as the particular galenic formulation, the mode of application, and the particular disease or condition to be treated and/or prevented. Other factors like age, body weight, sex, diet, time of administration, rate of excretion, condition of the host, drug combinations, reaction sensitivities and severity of the disease shall be taken into account. Administration can be carried out continuously or periodically within the maximum tolerated dose. Preferred doses for ibuprofen are from 15 mg/day to 1500 mg/day, more preferably from 100 mg/day to 1400 mg/day, still more preferably from 200 mg/day to 1300 mg/day, even more preferably from 200 mg/day to 1200 mg/day. Preferred doses for the Mg²⁺ source are from 200 mg/day to 350 mg/day, more preferably from 250 mg/day to 350 mg/day, even more preferably from 275 mg/day to 325 mg/day, still more preferably 300 mg/day. Preferred doses for the ascorbic acid or stereoisomer or pharmaceutically acceptable salt thereof are from 500 mg/day to 2000 mg/day, more preferably from 500 mg/day to 1500 mg/day, even more preferably from 750 mg/day to 1250 mg/day, still more preferably 1000 mg/day.

The combination for use according to the invention may be administered 1, 2, 3, 4 or 5 times/day, preferably, 1, 2, 3 or 4 times/day, more preferably 2 or 3 times/day. The combination for use according to the invention may be administered until the symptoms of the disease or conditions to be treated are reversed, alleviated, or their progress is inhibited. Preferably, the combination for use according to the present invention is administered for 3, 4, 5, 6 or 7 days, more preferably for 7 days.

The combination according to the invention may be administered by the oral, topical or parenteral routes; preferably by oral route.

### Pharmaceutical Compositions

In another aspect, the present invention relates to a pharmaceutical composition comprising a combination according to the present invention, i.e. which comprises ibuprofen, a Mg²⁺ source, and ascorbic acid or a stereoisomer or a pharmaceutically acceptable salt thereof, as described above, an a pharmaceutically acceptable excipient.

The term “pharmaceutically acceptable excipient” refers to a vehicle, diluent, or adjuvant that is administered with the active ingredient. Such pharmaceutical excipients can be sterile liquids, such as water and oils, including those of petroleum, animal, vegetable, or synthetic origin, such as peanut oil, soybean oil, mineral oil, sesame oil, and similars. Water or saline aqueous solutions and aqueous dextrose and glycerol solutions, particularly for injectable solutions, are preferably used as vehicles. Suitable pharmaceutical vehicles are described in “Remington's Pharmaceutical Sciences” by E. W. Martin, 21st Edition, 2005. Examples of pharmaceutically acceptable excipients for oral dosage pharmaceutical compositions of the invention are conventional excipients known in the art such as binding agents, for example syrup, acacia, gelatin, sorbitol, tragacanth, or polyvinylpyrrolidone; fillers, for example lactose, mannitol, xylitol, sorbitol, sucrose maize starch, calcium phosphate, sorbitol, glycine, dextrose, maltodextrin, dextran, dextrin, modified starches; glidants and tabletting lubricants, for example magnesium stearate, calcium stearate, zinc stearate stearic acid, glyceryl monostearate, glyceryl palmitostearate, hydrogenated castor oil, colloidal silicon dioxide, silicon dioxide, colloidal anhydrous silicon, glycerin, hydrogenated vegetable oil, mineral oil, polyethylene glycol, sodium benzoate, sodium lauryl sulfate, sodium stearyl fumarate, or talc; disintegrants, for example starch, polyvinylpyrrolidone, sodium starch glycollate, crospovidone, microcrystalline cellulose, hydroxypropyl cellulose, or sorbitan fatty acid esters; pharmaceutically acceptable wetting agents such as sodium lauryl sulfate; water solubilisation adjuvants such as urea, betain monohydrate, potassium sulphate, potassium acetate, mannitol; alkalizing agents such as, potassium carbonate, sodium carbonate, sodium bicarbonate, trisodium phosphate, tripotassium phosphate, trisodium citrate, tripotassium citrate; sweeteners such as sodium saccharine, sodium cyclamate and aspartame; flavouring agents such as menthol and mint oil.

The pharmaceutical compositions of the invention preferably contain arginine, e.g. in an amount of 5 to 10 wt. %.

The pharmaceutical compositions of the invention may additionally contain chelating agents such as ethylenediaminetetraacetic acid (EDTA), e.g. in an amount of 0.1 to 2 wt. %.

The pharmaceutical compositions of the invention may be administered by oral, topical or parenteral routes; preferably by oral route.

In a preferred embodiment the pharmaceutical compositions are in oral form, either solid or liquid. Suitable dose forms for oral administration may be tablets, capsules, syrups or solutions powder for oral solution or suspension, granules, sachet; preferably the dosage form is selected from the group consisting of powders, granules and sachets.

In a particularly preferred embodiment, the formulation comprises granules comprising ibuprofen and the Mg²⁺ source, and ascorbic acid as extragranular component. The granulation can be performed with standard granulation techniques, using either wet granulation or dry granulation. Wet granulation may be performed with e.g. water, ethanol, or isopropanol, with isopropanol being preferred. The preferred binder is povidone (polyvinyl pyrrolidone (PVP)). Besides ibuprofen and the Mg²⁺ source, the granules may contain other ingredients such as sodium bicarbonate and/or sucrose. To obtain the formulation of the present invention, ascorbic acid powder is added to the granules. In addition, other components may be added extracellularly, such as ingredients to improve taste (such as aroma (e.g. peppermint) or saccharin) and/or ingredients to improve flowability (such as silicon dioxide powder).

This formulation comprising granules and ascorbic acid powder is preferably provided in a sachet, more preferably in a vacuum sealed sachet.

In a particular embodiment, this sachet contains:
ibuprofen, its enantiomers or mixtures thereof, a hydrate or a pharmaceutically acceptable salt thereof (preferably ibuprofen lysinate or arginate, more preferably ibuprofen lysinate) in an amount of from 450 mg to 1050 mg expressed as equivalent ibuprofen acid,
a Mg2+ source selected from magnesium aspartate or a hydrate thereof (preferably magnesium aspartate dihydrate) or a combination of magnesium oxide and magnesium gluconate in an amount of from 75 mg to 225 mg expressed as Mg2+, and
L-ascorbic acid, a hydrate or a pharmaceutically acceptable salt thereof in an amount of from 300 mg to 900 mg expressed as equivalent free ascorbic acid.

The pharmaceutical compositions may also be adapted for parenteral administration, such as sterile solutions, suspensions or lyophilized products in the appropriate unit dosage form. Adequate excipients can be used, such as bulking agents, buffering agents or surfactants.

The pharmaceutical compositions may also be adapted for topical administration, such as sterile solutions, suspensions or lyophilized products in the appropriate unit dosage form. Adequate excipients can be used, such as bulking agents, buffering agents or surfactants. Examples of suitable dosage forms for topical administrations are gels, emulsions, solutions, creams, ointments, and the like. Pharmaceutical compositions for topical administration may also be applied on a carrier such as tapes, adhesive tapes, wraps, plasters, bandages, adhesive bandages, and the like.

The above mentioned formulations will be prepared using standard methods such as those described or referred to in the Spanish and US Pharmacopoeias and similar reference texts.

The following examples represent specific embodiments of the present invention. They do not intend to limit in any way the scope of the invention defined in the present description.

### EXAMPLES

### Example 1

### Pharmaceutical Combination Comprising Meloxicam+Mg+Vit C

### Reference Example

The pharmaceutical combination was prepared comprising the following ingredients:

Meloxicam 7.5 mg

Vitamin C 500 mg

Mg²⁺ 150 mg (Magnesium oxide 130 mg+Magnesium bromide 8 mg+Magnesium fluoride 0.35 mg)

This combination is suitable for administration twice a day.

### Example 2

### Treatment of TMJD with the Combination of Example 1

### Reference Example

The treatment was carried out in a group of 19 patients. All patients were diagnosed of TMDJ and received treatment A and treatment B in a blind crossover study (patients received a sequence of different treatments, i.e. treatment A and then treatment B, or treatment B and then treatment A) during four days:

A. treatment A comprises administration of the combination of Example 1 twice a day and the use of an occlusal splint for 8 hours during the night; and.

B. treatment B comprises administration of 7.5 mg meloxicam twice a day and the use of an occlusal splint for 8 hours during the night.

The results of the treatment were evaluated by taking into account the following six variables (likert items):

1. general pain relief;

2. relief of localized pain;

3. pain relief in comparison with other pharmacological treatments used for the management of TMDJ before this study, such as analgesics and muscular relaxants in combination with analgesics;

4. overall treatment efficacy;

5. treatment discomfort; and

6. quickness of relief onset.

Each patient is asked after each of the two treatments (A and B) to rate each of the six likert items using a scale from 1 to 5, wherein score 1 means very bad, score 2 means bad, score 3 means regular, score 4 means good and score 5 means very good.

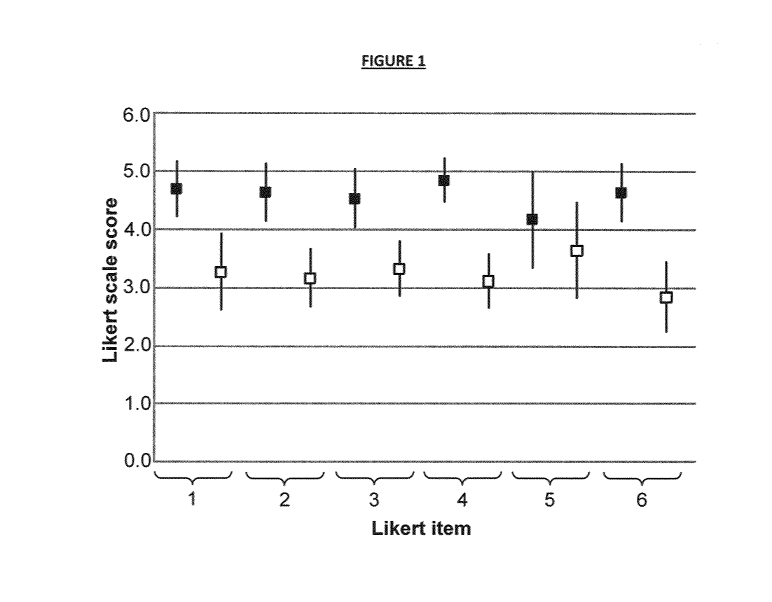

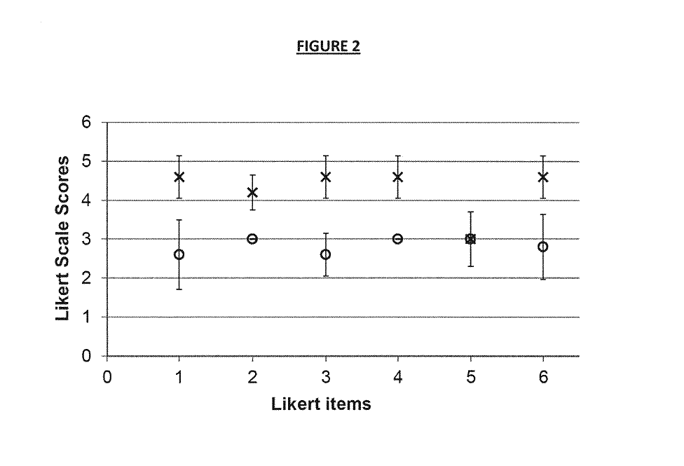

The mean of the likert scores of all the patients for each likert item was calculated and the results are shown in FIG. 1. As it can be seen, the combination of the COX inhibitor, vitamin C and the Mg²⁺ source (treatment A) was superior to the COX inhibitor alone (treatment B).

### Example 3

### Pharmaceutical Combination Comprising Ibuprofen, Magnesium and Vitamin C

The pharmaceutical combination comprises the following ingredients, to be administered T.I.D:

Ibuprofen 400 mg (racemic ibuprofen lysinate)

Vitamin C 330 mg

Mg²⁺100 mg (magnesium L-lactate)

The amount of ibuprofen refers to the equivalent amount of free ibuprofen present in the ibuprofen lysinate salt. The amount of Mg²⁺ refers to the amount of Mg²⁺ present in magnesium L-lactate.

This combination is suitable for administration three times a day.

### Example 4

### Pharmaceutical Combination Comprising Ibuprofen, Magnesium and Vitamin C

The pharmaceutical combination was prepared comprising the following:

a) Ibuprofen composition (1 tablet of a commercially available Ibuprofen Actron 400 mg composition):
ibuprofen 400 mg
PEG600 240.000 mg
potassium hydroxide 34.400 mg
purified water 45.600 mg
nipagin (methylparaben) 0.791 mg
nipasol (propylparaben) 0.198 mg
gelatin 213.576 mg
anhydrisorb 85/70 (D-sorbitol and 1,4-sorbitans) 104.217 mg
sunset yellow 0.049 mg

b) Mg2+ source composition (½ tablet of a commercially available GNC Magnesium 250 mg composition):
Mg2+125 mg (magnesium oxide+magnesium gluconate)
cellulose
titanium dioxide
vegetable acetoglycerides

c) vitamin C composition (3 tablets of a commercially available Esvit-C 100 mg composition):
vitamin C 300 mg

This combination is suitable for administration three times a day.

### Example 5

### Treatment of TMJD with the Combination of Example 4

The treatment was carried out in a group of 5 patients. All patients were diagnosed of TMDJ and received treatment A and treatment B in a blind crossover study (patients received a sequence of different treatments, i.e. treatment A and then treatment B, or treatment B and then treatment A) during four days:

A. Treatment A comprises administration of the combination of Example 4 three times a day; and

B. Treatment B comprises administration of 400 mg of ibuprofen (ibuprofen composition described as item a) in Example 1) three times a day.

The results of the treatment were evaluated by taking into account the following six variables (likert items):

1. general pain relief;

2. relief of localized pain;

3. pain relief in comparison with other pharmacological treatments used for the management of TMDJ before this study, such as analgesics and muscular relaxants in combination with analgesics;

4. overall treatment efficacy;

5. treatment discomfort; and

6. quickness of relief onset.

Each patient is asked after each of the two treatments (A and B) to rate each of the six likert items using a scale from 1 to 5, wherein score 1 means very bad, score 2 means bad, score 3 means regular, score 4 means good and score 5 means very good. The mean of the likert scores of all the patients for each likert item was calculated and the results are shown in FIG. 1. As it can be seen, the combination of ibuprofen, vitamin C and the Mg2+ source (treatment A) was superior to ibuprofen alone (treatment B).

### Example 6

### Pharmaceutical Combination Comprising Ibuprofen, Magnesium and

### Vitamin C

The pharmaceutical combination comprises three different sachets having the ingredients shown in Tables 1-3:

TABLE 1
Composition of sachet 1 (ibuprofen)
Component Reference/source Percentage mg/unit

| | | | | | | 50 | |
|---|---|---|---|---|---|---|---|
| | Component | Grade/Batch | Percentage | mg/Unit | | units | |
| a. | Ascorbic Acid | | 8.3% | 330.0 | mg | 16.5 | g |
| b. | Ibuprofen Lysinate | IBL1405018 | 17.1% | 683.6 | mg | 34.2 | g |
| | Shasun | | | | | | |
| c. | Magnesium Aspartate | | 26.7% | 1068.3 | mg | 53.4 | g |
| d. | Sodium Bicarbonate | | 20.0% | 800.0 | mg | 40.0 | g |
| e. | Sucrose | Di-Pac | 18.7% | 748.1 | mg | 37.4 | g |
| f. | Arginine | | 7.5% | 300.0 | mg | 15.0 | g |
| g. | Saccharin | | 0.6% | 25.0 | mg | 1.3 | g |
| h. | Novamint Fresh | 506041 TP0504 | 0.6% | 25.0 | mg | 1.3 | g |
| | Peppermint | (Firmenich) | | | | | |
| i. | Silicon dioxide | Aerosil pH 200 | 0.5% | 20.0 | mg | 1.0 | g |
| Total: | | | 100.0% | 4000.0 | mg | 200.0 | g |
| a. | Ascorbic Acid | | 8.3% | 330.0 | mg | 16.5 | g |
| b. | Ibuprofen Lysinate | IBL1405018 | 17.1% | 683.6 | mg | 34.2 | g |
| | Shasun | | | | | | |
| c. | Magnesium Aspartate | | 26.7% | 1068.3 | mg | 53.4 | g |
| d. | Sucrose | Di-Pac | 38.7% | 1548.1 | mg | 77.4 | g |
| e. | Arginine | | 7.5% | 300.0 | mg | 15.0 | g |
| f. | Saccharin | | 0.6% | 25.0 | mg | 1.3 | g |
| g. | Novamint Fresh | 506041 TP0504 | 0.6% | 25.0 | mg | 1.3 | g |
| | Peppermint | (Firmenich) | | | | | |
| h. | Silicon dioxide | Aerosil pH 200 | 0.5% | 20.0 | mg | 1.0 | g |
| Total: | | | 100.0% | 4000.0 | mg | 200.0 | g |
| a. | Ascorbic Acid | | 8.3% | 330.0 | mg | 16.5 | g |
| b. | Ibuprofen Lysinate | IBL1405018 | 17.1% | 683.6 | mg | 34.2 | g |
| | Shasun | | | | | | |
| c. | Magnesium Aspartate | | 26.7% | 1068.3 | mg | 53.4 | g |
| d. | EDTA | | 0.5% | 20.0 | mg | 1.0 | g |
| e. | Sucrose | Di-Pac | 38.2% | 1528.1 | mg | 76.4 | g |
| f. | Arginine | | 7.5% | 300.0 | mg | 15.0 | g |
| g. | Saccharin | | 0.6% | 25.0 | mg | 1.3 | g |
| h. | Novamint Fresh | 506041 TP0504 | 0.6% | 25.0 | mg | 1.3 | g |
| | Peppermint | (Firmenich) | | | | | |
| i. | Silicon dioxide | Aerosil pH 200 | 0.5% | 20.0 | mg | 1.0 | g |

| Total: | | 100.0% | 4000.0 | mg | 200.0 | g |
|---|---|---|---|---|---|---|
| | 100.0% | | | | | |

The amount of ibuprofen refers to the equivalent amount of free ibuprofen present in the ibuprofen lysinate salt. The amount of Mg2+ refers to the amount of Mg2+ present in magnesium aspartate dihydrate.

This combination is suitable for administration three times a day.

### Example 7

### Pharmaceutical Combination Comprising Ibuprofen, Magnesium and

### Vitamin C

A formulation was prepared with the following ingredients:

| | Component | Grade/Batch | Percentage | mg/Unit | |
|---|---|---|---|---|---|
| a. | Ibuprofen Lysinate | IBL1405018 | 17.1% | 683.6 | mg |
| | Shasun | | | | |
| b. | Magnesium Aspartate | | 26.7% | 1068.3 | mg |
| c. | Ascorbic Acid | | 8.3% | 330.0 | mg |
| d. | Sodium Bicarbonate | | 20.0% | 800.0 | mg |
| e. | Sucrose | Di-Pac | 23.2% | 928.1 | mg |
| f. | PVP K25 | Kollidon K25 | 3.0% | 120.0 | mg |
| g. | Saccharin | | 0.6% | 25.0 | mg |
| h. | Novamint Fresh | 506041 TP0504 | 0.6% | 25.0 | mg |
| | Peppermint | (Firmenich) | | | |
| i. | Silicon dioxide | Aerosil pH 200 | 0.5% | 20.0 | mg |
| j. | Isopropanol | | — | 500.0 | mg |

| | | | |
|---|---|---|---|
| Total: | 100.0% | 4000.0 | mg |

This batch was manufactured in a Rotolab High Shear mixer.

The intragranular components are ibuprofen, magnesium aspartate, sodium bicarbonate and sucrose. The granulation solution used is PVP K25 in isopropanol.

The extragranular components are ascorbic Acid, saccharin, aroma and silicon dioxide.

The flowability results obtained for this batch are:

| Funnel | Angle | Apparent | Tapped | Carr's | Haussner |
|---|---|---|---|---|---|
| Flow | Repose | Density | Density | Index | Ration |
| — | — | 0.661 g/ml | 0.778 g/ml | 15.00 | 1.18 |

### Example 8

### Global Pain Reduction in TMJ Patients

In order to test the role of vitamin C in the formulation 7 groups of patients (n=3) with temporomandibular pain were administered the following combinations:

Group 1 Ibu 300 Mg 80 vit c 200

Group 2 Ibu 300 Mg 80 vit C 600

Group 3 Ibu 600 Mg 80 vit C 200

Group 4 Ibu 600 Mg 80 vit C 600

Group 5 Ibu 300 Mg 80 vit C 60

Group 6 Ibu 600 Mg 80 vit C 60

Group 7 Ibu 0 Mg 0 vit C 600

Patients were treated 3 times over 24 h, and were asked to evaluate their pain (localized and general) 8 hours after the last administration using a visual analogic scale (VAS) from 1 to 10, being 10 the most positive situation. The results of the two assessments were summed up to obtain the global pain score.

### Results

| | Ibuprofen | Vit C | Mg | Reduction of |
|---|---|---|---|---|
| Group | (mg) | (mg) | (mg) | global Pain |
| 4 | 600 | 600 | 80 | 5.50 |
| 3 | 600 | 200 | 80 | 6.67 |
| 6 | 600 | 60 | 80 | −0.11 |
| 2 | 300 | 600 | 80 | 6.71 |
| 1 | 300 | 200 | 80 | 7.14 |
| 5 | 300 | 60 | 80 | 2.74 |
| 7 | 0 | 600 | 0 | 1.36 |

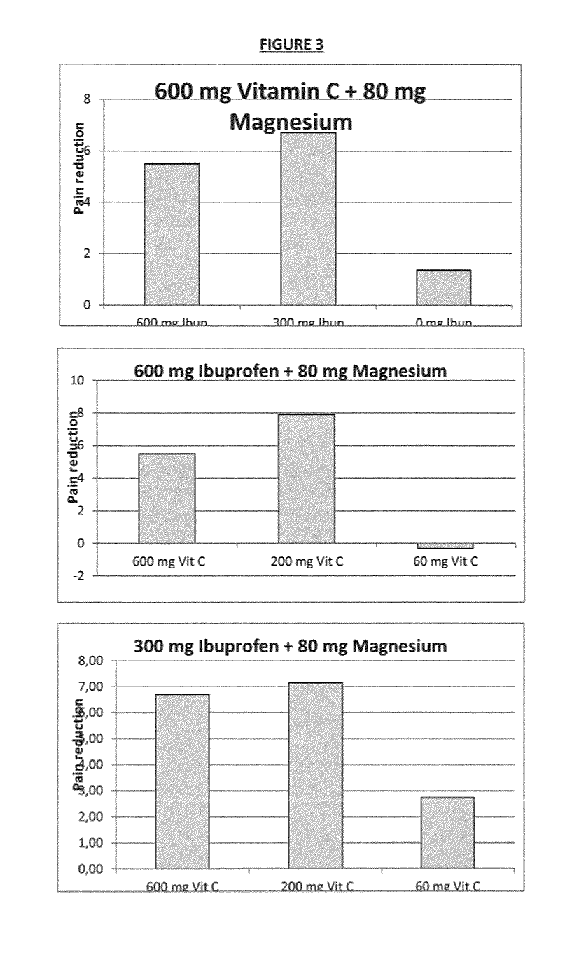

The results are also summarized in FIG. 3.

### CONCLUSIONS

1) Vit C without Mg and Ibuprofen has no effect. The group 7 has the worst values in all parameters

2) The groups 5, and 6 with dose of ratios of vitamin C to magnesium below 1:1 do not work in the reduction of pain in the conditions of the experiment.

3) Vitamin C at 600 mg and 200 mg in combination with ibuprofen (600 and 300 mg) works in the reduction of global pain. The effect is very high in both groups.

4) The main conclusion is that there is an unexpected potentiation effect of Vitamin C on the effect of combinations of Ibuprofen and Magnesium at doses of Vitamin C higher than 200 mg.

### Example 9

### Sport-Derived Muscle Stress/Pain Management

A combination of 7.5 mg meloxicam+500 mg Vitamin C+120 mg magnesium was administered to 35 elite rugby players. The treatment was applied to players with serious muscle strain and perception of potential muscle injury immediately after regular training or at post-game kinesics management, plus two more times at 12 and 24 h. All players reported feeling a more accelerated recovery compared to previous related situations without treatment, and a significant reduction in discomfort. None reported worsening of the potential lesion.

1. A combination comprising:
a) a COX inhibitor;
b) a Mg2+ source; and
c) ascorbic acid or a stereoisomer or a pharmaceutically acceptable salt thereof,
wherein the amount by weight of the COX inhibitor to the amount by weight of the Mg2+ source, expressed as Mg2+), is in the range from 0.04:1 to 15:1, and the amount by weight of ascorbic acid, or the stereoisomer or the pharmaceutically acceptable salt thereof, to the amount by weight of the Mg2+ source, expressed as Mg2+), is in the range from 1:1 to 12:1.

2. The combination according to claim 1, wherein when the Mg2+ source is selected from the group consisting of magnesium stearate, magnesium trisilicate, magnesium carbonate, magnesium oxide and magnesium hydroxide, then the combination comprises at least one further Mg2+ source which is not selected from the group consisting of magnesium stearate, magnesium trisilicate, magnesium carbonate, magnesium oxide and magnesium hydroxide.

3. The combination according to claim 1, wherein the COX inhibitor is selected from the group consisting of ibuprofen, meloxicam, aspirin, diflunisal, salsalate, diclofenac, naproxen, indomethacin, tolmetin, dexibuprofen, fenoprofen, mefenamic acid, ketoprofen, dexketoprofen, flurbiprofen, loxoprofen, oxaprozin, ketorolac, sulindac, etodolac, diclofenac, tenoxicam, piroxicam, paracetamol, ampiroxicam, bufexamac, zaltoprofen, flunixin meglumine, licofelone, lornoxicam, tolfenamic acid, acemetacin, triflusal, nimesulide, celecoxib, parecoxib, rofecoxib, valdecoxib, lumiracoxib, ceracoxib, etoricoxib, aceclofenac, their enantiomers, pharmaceutically acceptable salts and mixtures thereof.

4. The combination according to claim 3, wherein the COX inhibitor is ibuprofen, its enantiomers or mixtures thereof, and pharmaceutically acceptable salts thereof.

5. The combination according to claim 1, wherein the Mg2+ source is an organic magnesium salt.

6. The combination according to claim 5, wherein the organic magnesium salt is selected from the group consisting of magnesium aspartate, magnesium gluconate, magnesium citrate, magnesium pidolate, magnesium lactate, magnesium acetate, magnesium glycerophosphate, mixtures thereof and hydrates thereof.

7. The combination according to claim 1, wherein ascorbic acid, or a stereoisomer or a pharmaceutically acceptable salt thereof, is L-ascorbic acid, or a pharmaceutically acceptable salt thereof.

8. The combination according to claim 1, which comprises ibuprofen, its enantiomers or mixtures thereof, or a pharmaceutically acceptable salt thereof, as component a), magnesium lactate or magnesium aspartate as component b), and L-ascorbic acid, or a pharmaceutically acceptable salt thereof, as component c).

9. The combination according to claim 1, wherein the amount by weight of the COX inhibitor to the amount by weight of the Mg2+ source is in the range from 3:1 to 5.5:1, and the amount by weight of ascorbic acid, or the stereoisomer or the pharmaceutically acceptable salt thereof, to the amount by weight of the Mg2+ source is in the range from 1:1 to 5:1.

10. The combination according to claim 1, wherein the COX inhibitor, the Mg2+ source and ascorbic acid, or the stereoisomer or the pharmaceutically acceptable salt thereof, form part of the same composition.

11. The combination according to claim 1, wherein the COX inhibitor is ibuprofen, and wherein the amount by weight of ibuprofen to the amount by weight of the Mg2+ source is in the range from 2:1 to 6:1, and the amount by weight of ascorbic acid, or the stereoisomer or the pharmaceutically acceptable salt thereof, to the amount by weight of the Mg2+ source is in the range from 1:1 to 5:1.

12. The combination according to claim 1, wherein the COX inhibitor is ibuprofen, and wherein the amount by weight of ibuprofen to the amount by weight of the Mg2+ source is in the range from 4:1 to 6:1, and the amount by weight of ascorbic acid, or the stereoisomer or the pharmaceutically acceptable salt thereof, to the amount by weight of the Mg2+ source is in the range from 2:1 to 6:1.

13. The combination claim 10, wherein the composition is provided in a dosage form selected from sachets, tablets, granules, and powders.

14. The combination according to claim 13, wherein the dosage form adapted for a single administration comprises:
from 300 mg to 700 mg of the COX inhibitor,
from 50 mg to 150 mg of the Mg2+ source, expressed as Mg2+, and
from 200 mg to 600 mg of ascorbic acid, or the stereoisomer or the pharmaceutically acceptable salt thereof, expressed as equivalent ascorbic acid.

15. The combination according to claim 13, wherein the dosage form comprises granules comprising the COX inhibitor and the Mg2+ source, and ascorbic acid as an extragranular component.

16. The combination according to claim 15, wherein the dosage form is a sachet.

17. The combination according to claim 15, wherein the sachet is vacuum sealed.

18. (canceled)

19. A method for treatment or prevention, or both, of a disease or condition selected from the group consisting of temporomandibular joint disorder, pain accompanying muscle contracture, pain accompanying muscle overload following exercise and muscle pain during episodes of fibromyalgia, the method comprising administering an effect amount of the combination of claim 1 to a patient in need thereof.

20. The method according to claim 19, comprising administering the combination one, two or three times a day, at a daily dose of from 15 mg to 1500 mg of the COX inhibitor, from 200 mg to 350 mg of the Mg2+ source, and from 500 mg to 2000 mg of ascorbic, acid or a stereoisomer or a pharmaceutically acceptable salt thereof.

21. A pharmaceutical composition comprising the combination of claim 1 and a pharmaceutically acceptable excipient.
